(19) United States
(12) Patent Application Publication
(10) Pub. No.: US 2022/0117691 A1
(43) Pub. Date: Apr. 21, 2022

(54) ADAPTOR, METHOD OF DETACHING ADAPTOR FROM ROBOT ARM, AND ROBOTIC SURGICAL SYSTEM

(71) Applicant: MEDICAROID CORPORATION, Kobe-shi (JP)

(72) Inventors: Kenji AGO, Kobe-shi (JP); Koji MUNETO, Kobe-shi (JP); Kazuhiro SATO, Kobe-shi (JP)

(73) Assignee: MEDICAROID CORPORATION, Kobe-shi (JP)

(21) Appl. No.: 17/502,059

(22) Filed: Oct. 15, 2021

(30) Foreign Application Priority Data
Oct. 19, 2020 (JP) 2020-175590

(51) Int. Cl. A61B 90/00; A61B 34/30; A61B 46/10; A61B 17/00

(52) U.S. Cl. CPC A61B 90/08; A61B 34/30; A61B 2017/0046; A61B 17/00234; A61B 2017/00477; A61B 46/10

(57) ABSTRACT

An adaptor according one or more embodiments may include a base body and an arm engagement part including an engagement portion to be engaged with the robot arm at an engagement position and a contact portion with which a release tool comes in contact. The arm engagement part may be configured such that the contact portion comes in contact with the release tool when the release tool is inserted through the tool insertion hole, a part of the arm engagement part is moved into an escape space in the base body when the contact portion is moved in a direction orthogonal to an insertion direction of the release tool, and the engagement portion of the arm engagement part is moved, when the release tool is further inserted, from the engagement position to the disengagement position, which disengages the engagement portion of the arm engagement part from the robot arm.

### CROSS REFERENCE TO RELATED APPLICATIONS

This application claims priority to Japanese Patent Application No. 2020-175590 filed on Oct. 19, 2020, the entire contents of which are incorporated herein by reference.

### BACKGROUND

The disclosure may relate to an adaptor, a method of detaching an adaptor from a robot arm, and a robotic surgical system.

In a related art, there has been known a surgical instrument that is to be attached to a robot arm via an adaptor.

U.S. Pat. No. 9,839,487 discloses a surgical instrument that is to be attached to a robot arm via an adaptor. The surgical instrument is configured to be detached from the adaptor by inserting a release tool into an opening provided to a base body of the adaptor in a state where the surgical instrument is attached to the adaptor, moving the release tool in a direction orthogonal to an insertion direction of the release tool, and operating the release tool to release engagement between the adaptor and the surgical instrument.

### SUMMARY

However, the surgical instrument disclosed in U.S. Patent Application Publication No. 9,839,487 is configured to be detached from the adaptor by inserting the release tool into the opening provided to the base body of the adaptor in the state where the surgical instrument is attached to the adaptor, moving the release tool in the direction orthogonal to the insertion direction of the release tool, and operating the release tool to release the engagement between the adaptor and the surgical instrument. Accordingly, upon detaching the surgical tool from the robot arm, it is needed to insert the release tool and then move the release tool in the direction crossing the insertion direction of the release tool. Therefore, the work of detaching the surgical instrument from the robot arm may be complicated.

An object of one or more embodiments of the disclosure may be to prevent the work of detaching the surgical instrument from the robot arm from being complicated.

An adaptor according to a first aspect of the disclosure may be an adaptor to be detachably attached to a robot arm for transmitting a driving force from the robot arm to a surgical instrument. The adaptor may include: a base body including a first surface to be attached to the robot arm and formed with an opening and a second surface to which the surgical instrument is to be attached; an arm engagement part including an engagement portion configured to be movable between an engagement position corresponding to the opening of the first surface and a disengagement position retracted from the engagement position and to be engaged with the robot arm at the engagement position, and a contact portion with which a release tool comes in contact. The base body is formed with a tool insertion hole through which the release tool is to be inserted and an escape space into which a part of the arm engagement part is to be moved by the release tool that is inserted through the tool insertion hole and is in contact with the arm engagement part. The arm engagement part is configured such that the contact portion of the arm engagement portion comes in contact with the release tool when the release tool is inserted through the tool insertion, the part of the arm engagement part is move into the escape space when the contact portion is moved in a direction orthogonal to an insertion direction of the release tool, and the engagement portion of the arm engagement part is moved, when the release tool is further inserted, from the engagement position to the disengagement position so as to disengage the engagement portion of the arm engagement part from the robot arm.

A method according to a second aspect of the disclosure may be a method of detaching an adaptor from a robot arm, wherein the adaptor includes a base body including a first surface to be attached to the robot arm and a second surface to which the surgical instrument is to be attached, and an arm engagement part including an engagement portion to be engaged with the robot arm, and wherein the adaptor is configured to be detachably attached to the robot arm and transmit a driving force from the robot arm to the surgical instrument. The method may include: inserting a release tool for releasing engagement between the robot arm and the engagement portion of the arm engagement part of the adaptor into a tool insertion hole provided to the base body, to bring the release tool in contact with a contact portion of the arm engagement portion; moving the contact portion in a direction orthogonal to an insertion direction of the release tool to move a part of the arm engagement part to an escape space in the base body; and further inserting the release tool to move the engagement portion of the arm engagement part from an engagement position to a disengagement position so as to release the engagement between the robot arm and the engagement portion of the arm engagement part.

A robotic surgical system according to a third aspect of the disclosure may include: a surgical instrument; and an adaptor detachably attachable to a robot arm and configured to transmit a driving force from the robot arm to the surgical instrument. The adaptor may include: a base body including a first surface to be attached to the robot arm and formed with an opening and a second surface to which the surgical instrument is to be attached; an arm engagement part including an engagement portion configured to be movable between an engagement position corresponding to the opening of the first surface and a disengagement position retracted from the engagement position and to be engaged with the robot arm at the engagement position, and a contact portion with which a release tool comes in contact. The base body includes a tool insertion hole through which the release tool is to be inserted and an escape space into which a part of the arm engagement part is to be moved by the release tool that is inserted through the tool insertion hole and is in contact with the arm engagement part. The arm engagement part is configured such that the contact portion of the arm engagement part comes in contact with the release tool when the release tool is inserted through the tool insertion hole, the part of the arm engagement part is moved into the escape space when the contact portion is moved in a direction orthogonal to an insertion direction of the release tool, and the engagement portion of the arm engagement part is moved from the engagement position to the disengagement position when the release tool is further inserted, so as to disengage the engagement portion of the arm engagement part from the robot arm.

### BRIEF DESCRIPTION OF THE DRAWINGS

FIG. 1 is a diagram illustrating an overview of a robotic surgical system according to an embodiment;

FIG. 2 is a block diagram illustrating a view of a control-related configuration of the robotic surgical system according to an embodiment;

FIG. 3 is a diagram illustrating a perspective view of a state where a surgical instrument is attached to a robot arm via an adaptor according to an embodiment;

FIG. 4 is a diagram illustrating a perspective view of the adaptor and the surgical instrument according to an embodiment as seen from below;

FIG. 5 is a diagram illustrating a perspective view of the adaptor according to an embodiment as seen from above;

FIG. 6 is a diagram illustrating an exploded perspective view of a drive transmission member of the adaptor according to an embodiment;

FIG. 7 is a diagram illustrating a perspective view of a state where the adaptor is detached from a mount portion of the robot arm according to an embodiment;

FIG. 8 is a diagram illustrating a perspective view of an arm engagement part of the adaptor according to an embodiment;

FIG. 9 is a diagram illustrating a perspective view for explaining attachment of the adaptor to the robot arm according to an embodiment;

FIG. 10 is a diagram illustrating an explanatory view for explaining attachment of the surgical instrument to the adaptor that is attached to the robot arm according to an embodiment;

FIG. 11 is a diagram illustrating a view of a state where an engagement portion of the adaptor is moved to a disengagement position according to an embodiment;

FIG. 12 is a diagram illustrating a view of a state where restriction on movement of an operation portion by a stopper of the adaptor is released according to an embodiment;

FIG. 13 is a diagram illustrating a view of a state where the engagement portion of the adaptor is moved in an engagement position according to an embodiment;

FIG. 14 is a diagram illustrating a view of a state where the surgical instrument is attached to the robot arm via the adaptor;

FIG. 15 is a diagram illustrating a perspective view of a state where a release tool is inserted in a tool insertion hole of the adaptor according to an embodiment;

FIG. 16 is a diagram illustrating a view of a state where the release tool is inserted in the tool insertion hole of the adaptor according to an embodiment;

FIG. 17 is a diagram illustrating a view of a state where a part of the arm engagement part of the adaptor is moved in an escape space according to an embodiment; and

FIG. 18 is a diagram illustrating a view of a state where the engagement portion is moved in the disengagement position by the release tool that is inserted in the tool insertion hole of the adaptor according to an embodiment.

### DETAILED DESCRIPTION

Descriptions are provided hereinbelow for one or more embodiments based on the drawings.

### (Configuration of Robotic Surgical System)

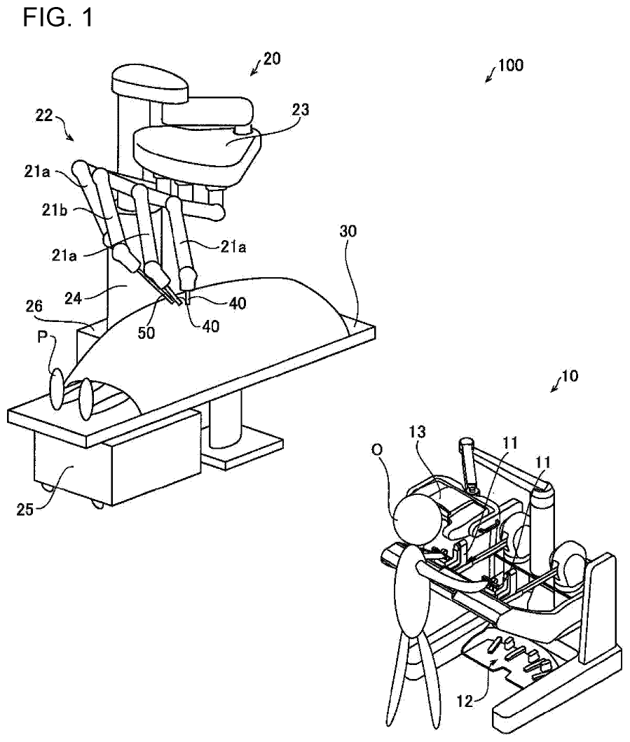

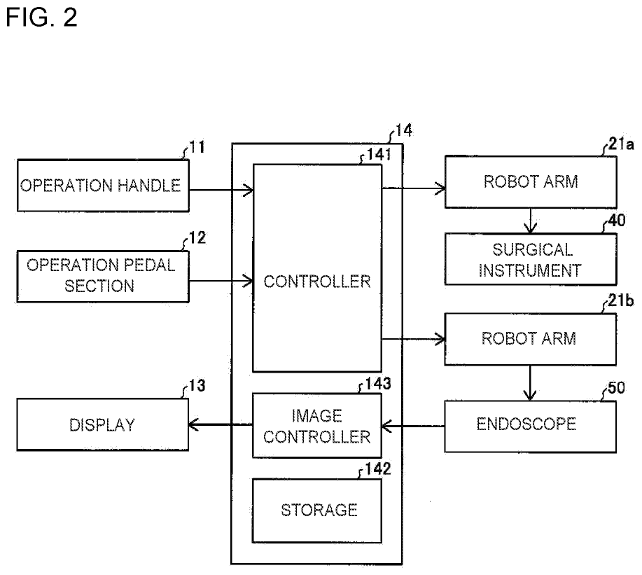

A configuration of a robotic surgical system 100 according to an embodiment is described with reference to FIGS. 1 and 2.

As illustrated in FIG. 1, the robotic surgical system 100 includes a remote control apparatus 10 and a patient-side apparatus 20. The remote control apparatus 10 is provided to remotely control medical equipment provided for the patient-side apparatus 20. When an operator O, as a surgeon, inputs an action mode instruction to be executed by the patient-side apparatus 20, to the remote control apparatus 10, the remote control apparatus 10 transmits the action mode instruction to the patient-side apparatus 20 through a controller 26. In response to the action mode instruction transmitted from the remote control apparatus 10, the patient-side apparatus 20 operates the medical equipment such as surgical instruments 40, an endoscope 50, and the like, attached to robot arms 21a. This allows minimally invasive surgery.

The patient-side apparatus 20 constitutes an interface to perform a surgery for a patient P. The patient-side apparatus 20 is positioned beside an operation table 30 on which the patient P is laid. The patient-side apparatus 20 includes plural robot arms 21a and 21b. One (21b) of the robot arms holds the endoscope 50 and the other robot arms (21a) hold the surgical instruments 40. The robot arms 21a and 21b are commonly supported by a platform 23. Each of the plural robot arms 21a and 21b includes plural joints. Each joint includes a driver provided with a servo-motor and a position detector such as an encoder. The robot arms 21a and 21b are configured so that the medical equipment attached to each of the robot arms 21a and 21b is controlled by a driving signal given through the controller 26 and performs a desired movement.

The platform 23 is supported by a positioner 22 placed on the floor of an operation room. The positioner 22 includes a column 24 and a base 25. The column 24 includes an elevating shaft adjustable in the vertical direction. The base 25 includes wheels and is movable on the floor surface.

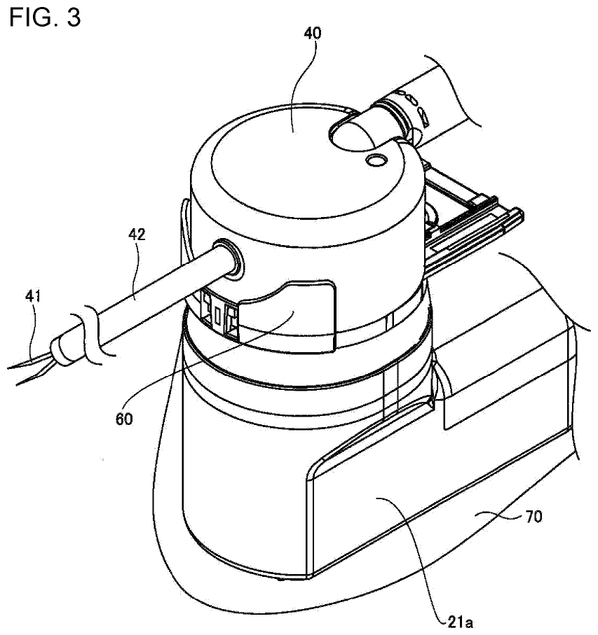

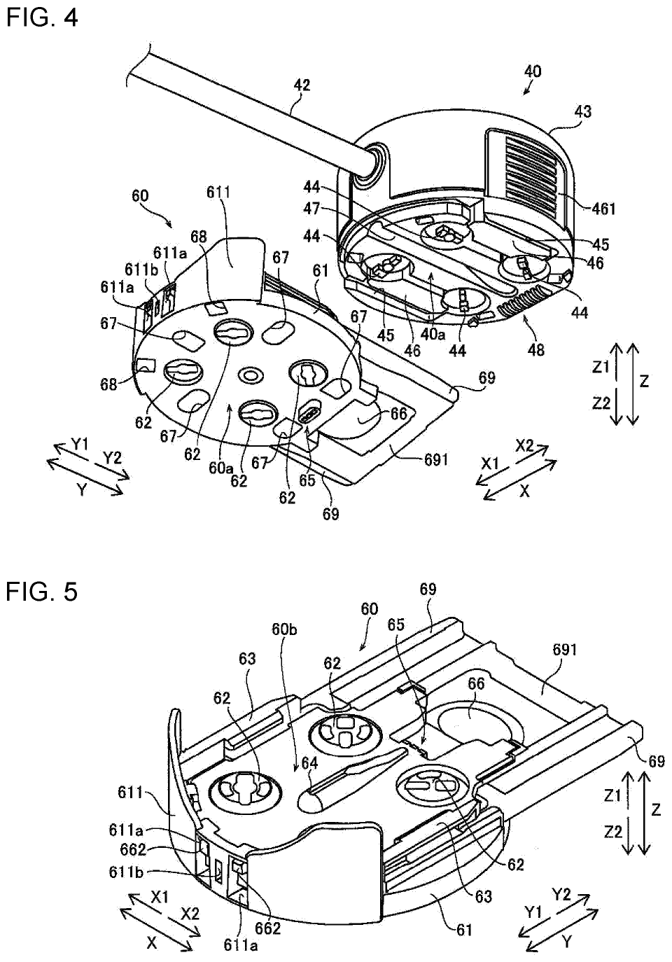

The surgical instruments 40 as the medical equipment are detachably attached to the distal ends of the robot arms 21a. Each surgical instrument 40 includes: a housing 43 (see FIG. 4), which is attached to the robot arm 21a; an elongated shaft 42 (see FIG. 4); and an end effector 41 (see FIG. 3), which is provided at the tip of the shaft 42. The end effector 41 may be grasping forceps, scissors, a hook, a high-frequency knife, a snare wire, a clamp, or a stapler, for example. The end effector 41 is not limited to those and can be various types of treatment tools. In surgeries using the patient-side apparatus 20, the robot arms 21a introduce the surgical instruments 40 into the body of the patient P through a cannula (trocar) placed on the body surface of the patient P. The end effectors 41 of the surgical instruments 40 are then located near a surgery site.

To the distal end of the robot arm 21b, the endoscope 50 as the medical equipment is detachably attached. The endoscope 50 captures an image in a body cavity of the patient P. The captured image is outputted to the remote control apparatus 10. The endoscope 50 is a 3D endoscope capable of capturing a three-dimensional image or a 2D endoscope. In surgeries using the patient-side apparatus 20, the robot arm 21b introduces the endoscope 50 into the body of the patient P through a trocar placed on the body surface of the patient P. The endoscope 50 is then located near the surgery site.

The remote control apparatus 10 constitutes the interface with the operator O. The remote control apparatus 10 is an apparatus that allows the operator O to operate the medical equipment attached to the robot arms 21a. Specifically, the remote control apparatus 10 is configured to transmit action mode instructions which are inputted by the operator O and are to be executed by the surgical instruments 40 and endoscope 50, to the patient-side apparatus 20 through the controller 26. The remote control apparatus 10 is installed beside the operation table 30 so that the operator O can see the condition of the patient P very well while operating the remote control apparatus 10, for example. The remote control apparatus 10 may be configured to transmit action mode instructions wirelessly and be installed in a room different from the operation room where the operation table 30 is installed.

The action modes to be executed by the surgical instruments 40 include modes of actions to be taken by each surgical instrument 40 (a series of positions and postures) and actions to be executed by the function of each surgical instrument 40. When the surgical instrument 40 is a pair of grasping forceps, for example, the action modes to be executed by the surgical instrument 40 include roll and pitch positions of the wrist of the end effector 41 and actions to open and close the jaws. When the surgical instrument 40 is a high-frequency knife, the action modes to be executed by the surgical instrument 40 include vibration of the high-frequency knife, specifically, supply of current to the high-frequency knife. When the surgical instrument 40 is a snare wire, the action modes to be executed by the surgical instrument 40 include a capturing action and an action to release the captured object. Further, the action modes may include an action to supply current to a bipolar or monopolar instrument to burn off the surgery site.

The action modes to be executed by the endoscope 50 include the position and posture of the tip of the endoscope 50 and setting of the zoom magnification, for example.

As illustrated in FIGS. 1 and 2, the remote control apparatus 10 includes operation handles 11, an operation pedal section 12, a display 13, and a control apparatus 14.

The operation handles 11 are provided in order to remotely operate medical equipment attached to the robot arms 21a. Specifically, the operation handles 11 accept operations by the operator O for operating the medical equipment (the surgical instruments 40 and endoscope 50). The operation handles 11 are composed of two operation handles 11 arranged side by side in the horizontal direction. One of the two operation handles 11 is operated by the right hand of the operator O while the other of the two operation handle 11 is operated by the left hand of the operator O.

The operation handles 11 extend from the rear side of the remote control apparatus 10 toward the front side. The operation handles 11 are configured to move in a predetermined three-dimensional operation region. Specifically, the operation handles 11 are configured so as to move up and down, right and left, and forward and rearward.

The remote control apparatus 10 and patient-side apparatus 20 constitute a master-slave system in terms of controlling movement of the robot arms 21a and robot arm 21b. The operation handles 11 constitute an operating part on the master side in the master-slave system, and the robot arms 21a and 21b holding the medical equipment constitute an operating part on the slave side. When the operator O operates the operation handles 11, the movement of one of the robot arms 21a or 21b is controlled so that the distal end portion (the end effector 41 of the surgical instrument 40) of the robot arm 21a or the distal end portion (the endoscope 50) of the robot arm 21b moves following the movement of the operation handles 11.

The patient-side apparatus 20 controls the movement of the robot arms 21a in accordance with the set motion scaling ratio. When the motion scaling ratio is set to ½, for example, the end effectors 41 of the surgical instruments 40 move ½ of the movement distance of the operation handles 11. This allows for precise fine surgery.

The operation pedal section 12 includes plural pedals to execute medical equipment-related functions. The plural pedals include a coagulation pedal, a cutting pedal, a camera pedal, and a clutch pedal. The plural pedals are operated by a foot of the operator O.

The coagulation pedal enables the surgical instrument 40 to coagulate a surgery site. Specifically, when the coagulation pedal is operated, voltage for coagulation is applied to the surgical instrument 40 to coagulate the surgery site. The cutting pedal enables the surgical instrument 40 to cut the surgery site. Specifically, the cutting pedal is operated to apply voltage for cutting to the surgical instrument 40 and cut a surgery site.

The camera pedal is used to control the position and orientation of the endoscope 50 that captures images within the body cavity. Specifically, the camera pedal enables operation of the endoscope 50 by the operation handles 11. That is, the position and orientation of the endoscope 50 are controllable by the operation handles 11 while the camera pedal is being pressed. The endoscope 50 is controlled by using both of the right and left operation handles 11, for example. Specifically, when the operator O rotates the right and left operation handles 11 about the middle point between the right and left operation handles 11, the endoscope 50 is rotated. When the operator O presses the right and left operation handles 11 together, the endoscope 50 goes forward into the body cavity. When the operator O pulls the right and left operation handles 11 together, the endoscope 50 goes back. When the operator O moves the right and left operation handles 11 together up, down, right, or left, the endoscope 50 moves up, down, right, or left, respectively.

The clutch pedal is used to temporarily disconnect operation-related connection between the operation handles 11 and the robot arms 21a to stop movement of the surgical instruments 40. Specifically, when the clutch pedal is being pressed, the robot arms 21a of the patient-side apparatus 20 do not work even if the operation handles 11 are operated. For example, when the operation handles 11 are operated and moved to the edge of the range of movement, the operator O operates the clutch pedal to temporarily disconnect the operation-related connection and then returns the operation handles 11 to the center of the range of movement. When the operator O stops operating the clutch pedal, the operation handles 11 are again connected to the robot arms 21a. The operator O restarts the operation for the operation handles 11 around the center thereof.

The display 13 (or a display device) is configured to display images captured by the endoscope 50. The display 13 includes a scope type display or a non-scope type display. The scope type display is a display that the operator O looks into. The non-scope type display is a display like an open-type display that includes a flat screen and the operator is able to see without looking into, such as normal displays for personal computers.

When the scope type display is attached, the scope type display displays 3D images captured by the endoscope 50 attached to the robot arm 21b of the patient-side apparatus 20. When the non-scope type display is attached, the non-scope type display also displays 3D images captured by the endoscope 50 provided for the patient-side apparatus 20. The non-scope type display may display 2D images captured by the endoscope 50 provided for the patient-side apparatus 20.

As illustrated in FIG. 2, the control apparatus 14 includes a controller 141, a storage 142, and an image controller 143, for example. The controller 141 includes a calculator such as a CPU. The storage 142 includes a memory, such as a ROM (Read Only Memory) and a RAM (Random Access Memory). The control apparatus 14 may be composed of a single controller performing centralized control or may be composed of plural controllers that perform decentralized control in cooperation with each other. The controller 141 determines whether an action mode instruction inputted by the operation handles 11 is to be executed by the robot arms 21a or to be executed by the endoscope 50, depending on the state of the operation pedal section 12. When determining that the action mode instruction inputted by the operation handles 11 is to be executed by any one of the surgical instruments 40, the controller 141 transmits the action mode instruction to the corresponding robot arm 21a. The robot arm 21a is thereby driven for controlling movement of the surgical instrument 40 attached to the robot arm 21a.

When determining that the action mode instruction inputted by the operation handles 11 is to be executed by the endoscope 50, the controller 141 transmits the action mode instruction to the robot arm 21b. The robot arm 21b is thereby driven for control of movement of the endoscope 50 attached to the robot arm 21b.

The storage 142 stores control programs corresponding to the types of the surgical instrument 40, for example. The controller 141 reads the stored control programs according to the types of the attached surgical instruments 40. The action mode instructions from the operation handles 11 and/or the operation pedal section 12 of the remote control apparatus 10 thereby cause the respective surgical instruments 40 to perform proper movements.

The image controller 143 transmits images acquired by the endoscope 50 to the display 13. The image controller 143 performs processing and modifying the images when needed.

### (Configurations of Adaptor and Surgical Instrument)

With reference to FIGS. 3 to 18, configurations of an adaptor 60 and the surgical instrument 40 according to an embodiment are described.

As illustrated in FIG. 3, the robot arm 21a (21b) is used in a clean area and is covered with a drape 70. In operation rooms, clean technique is used in order to prevent surgical incision sites and the medical equipment from being contaminated by pathogen, foreign matters, or the like. The clean technique defines a clean area and a contaminated area, which is outside the clean area. The surgery sites are located in the clean area. Members of the surgical team, including the operator O, make sure that only sterile objects are placed in the clean area during surgery and perform sterilization for an object which is to be moved to the clean area from the contaminated area. Similarly, when the members of the surgical team including the operator O place their hands in the contaminated area, the members sterilize their hands before directly touching objects located in the clean area. Instruments used in the clean area are sterilized or are covered with sterile drape 70.

The drape 70 is arranged between the robot arm 21a and the surgical instrument 40. Specifically, the drape 70 is arranged between the adaptor 60 and the robot arm 21a. The adaptor 60 is attached to an attachment portion 211 of the robot arm 21a with the drape 70 therebetween. Specifically, the adaptor 60 is a drape adaptor that puts the drape 70 between the adaptor 60 and the robot arm 21a. The surgical instrument 40 is attached to the adaptor 60 . The robot arm 21a transmits driving force to the surgical instrument 40 through the adaptor 60 to drive the end effector 41 of the surgical instrument 40.

As illustrated in FIGS. 4 and 5, the adaptor 60 includes a base body 61, drive transmission members 62, guide rails 63, a precedence guide rail 64, an electrode array 65, and an arm engagement part 66 (or an arm engagement member 66). As illustrated in FIG. 4, the adaptor 60 includes plural openings 67 and positioning holes 68. In addition, the adaptor 60 includes precedence guide portions 69. The adaptor 60 includes a first surface 60a that is arranged in the Z2 side and to which the robot arm 21a is attached. The adaptor 60 includes a second surface 60b that is arranged in the Z1 side and to which the surgical instrument 40 is attached.

As illustrated in FIG. 4, an attachment surface 40a arranged in the Z2 side of the housing 43 of the surgical instrument 40 is attached to the adaptor 60. The surgical instrument 40 includes: plural driven members 44; two guide grooves 45; two movable members 46; a precedence guide groove 47; and an electrode array 48.

As illustrated in FIG. 7, the adaptor 60 is attached to the attachment portion 211 of the robot arm 21a. The robot arm 21a includes plural drive members 212, engagement projections 213a, 213b, and 213c, and bosses 214. The engagement projections 213b are composed of two engagement projections 213b and the engagement projections 213c are composed of two engagement projections 213c, respectively.

Each of the engagement projections 213a, 213b, and 213c includes a groove 2131. The engagement projections 213a, 213b, and 213c are provided to be engaged with the adaptor 60. That is, engagement portions 661a, 661b, and 661c of the adaptor 60 are engaged with the grooves 2131 of the engagement projections 213a, 213b, and 213c of the robot arm 21a, respectively. Each of the grooves 2131 is opened toward the Y2 direction.

As illustrated in FIG. 4, the driven members 44 of the surgical instrument 40 are driven to rotate to drive the end effector 41. Specifically, the driven members 44 are connected to the end effector 41 with wires passing through the inside of the shaft 42. With the driven members 44 being rotated, the wires 421 are drawn so that the end effector 41 is driven. In the housing 43, one of the driven members 44 is connected to the shaft 42 through a gear(s). With the one of driven members 44 being rotated, the shaft 42 is rotated.

For example, the driven member 44 are composed of four driven members 44. The shaft 42 is rotated by the rotation of one of the driven members 44. The end effector 41 is driven by the rotations of the other three of the driven members 44. The four driven members 44 are arranged in two rows in the X direction and two columns in the Y direction.

Each of the guide grooves 45 is provided to extend along the Y direction. The guide grooves 45 are composed of two guide grooves 5 opposed to each other in the X direction. The two guide grooves 45 are provided substantially parallel to each other. The guide grooves 45 are respectively inserted to the guide rails 63 of the adaptor 60, to guide attachment of the surgical instrument 40 to the adaptor 60. Specifically, the width of each guide groove 45 is varied according to movement in the X direction of the corresponding movable member 46. That is, when the movable member 46 is moved inward, the width of the guide groove 45 is increased. When the movable member 46 is moved outward, the width of the guide groove 45 is decreased. Each of the movable members 46 is biased to a direction (an outward direction) in which the width of the guide groove 45 is decreased. Specifically, each of the movable members 46 is biased by a spring. The movable members 46 are moved in directions (inward directions) in which the widths of the guide grooves 45 are increased when an operator presses buttons 461.

The precedence guide groove 47 is provided to extend along the Y direction. The precedence guide groove 47 is provided between the two guide grooves 45. The precedence guide groove 47 is formed to extend substantially parallel to the two guide grooves 45. The precedence guide groove 47 is provided in the substantial center in the X direction of the attachment surface 40a.

The electrode array 48 is connected to the robot arm 21a through the electrode array 65 of the adaptor 60. The electrode array 48 is connected to a board provided in the housing 43. Specifically, the board of the surgical instrument 40 is connected to the robot arm 21a by attaching the surgical instrument 40 to the robot arm 21a via the adaptor 60. The board in the housing 43 is used for, for example, managing types of the surgical instrument 40 and the number of times the surgical instrument 40 is used.

As illustrated in FIGS. 4 and 5, the adaptor 60 is provided for detachably connecting the surgical instrument 40 to the robot arm 21a of the robotic surgical system 100. The base body 61 of the adaptor 60 includes the first surface 60a to be attached to the robot arm 21a and the second surface 60b to which the attachment surface 40a of the surgical instrument 40 is mounted. The adaptor 60 is configured to be detachably attached to the robot arm 21. The adaptor 60 is configured to transmit the driving force from the robot arm 21a to the surgical instrument 40.

The base body 61 is provided with a contact portion 611 which is to be in contact with the surgical instrument 40. The contact portion 611 is provided in the vicinity of an end portion on the Y1 side of the second surface 60b of the base body 61. The contact portion 611 is formed to protrude toward the surgical instrument 40 side (the Z1 side). The contact portion 611 is configured to be in contact with the Y1-side end of the housing 43 of the surgical instrument 40 in a state where the surgical instrument 40 is attached to the adaptor 60.

Openings 611a and a tool insertion hole 611b are provided in a central portion of the contact portion 611 in the X direction. The openings 611a are configured such that restriction portions 662 of the arm engagement part 66 enter in the openings 611a in a state where the engagement portions 661a to 661c of the arm engagement part 66 are located at the engagement position. The openings 611a are composed of a pair of openings 611a arranged in the X direction. The pair of openings 611a are arranged with the tool insertion hole 611b between the pair of openings 611a.

The tool insertion hole 611b is provided for inserting therein a release tool 80 (see FIG. 15). Specifically, the tool insertion hole 611b is provided in which the release tool 80 (see FIG. 15) is inserted to move the arm engagement part 66, for a case where the user wants to remove the surgical instrument 40 from the robot arm 21 but cannot remove the surgical instrument 40 from the adaptor 60 and cannot operate an operating portion 663 (a grip portion or a manipulation portion) of the arm engagement part 66. The case where the surgical instrument 40 cannot be removed from the adaptor 60 may include a case where the movable member 46 does not move even if the button 461 of the surgical instrument 40 is pressed, a case where the robot arm 21a or the like interferes with the button 461 of the surgical instrument 40 and thus the button 461 cannot be pressed, and the like. The case where the operating portion 663 of the arm engagement part 66 cannot be operated may include a case where an electrode terminal is provided on the Y2 side of the surgical instrument 40 and thus the operating portion 663 cannot be gripped, a case where the robot arm 21a interferes with the operating portion 663 and thus the operating portion 663 cannot be gripped, and the like.

As illustrated in FIG. 5, the tool insertion hole 611b is formed in a slit shape extending in the direction (the Z direction) in which the first surface 60a and the second surface 60b are opposed. With this configuration, it is possible to prevent a finger or the like from entering through the tool insertion hole 611b and thus to prevent unintentionally pressing the contact portion 665 of the arm engagement part 66 with a finger.

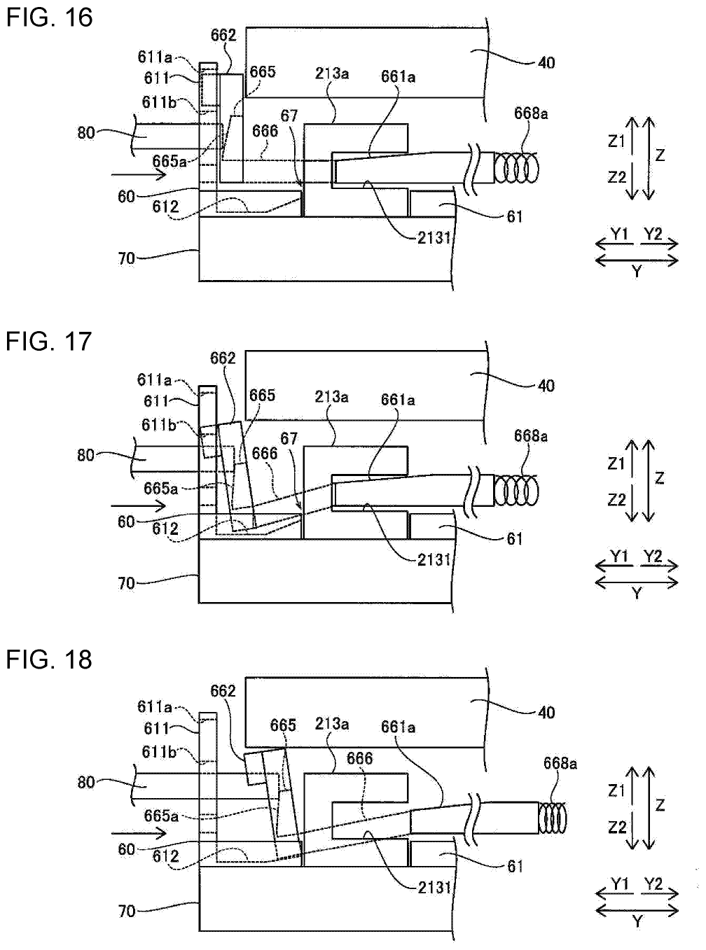

As illustrated in FIGS. 16 to 18, the base body 61 is formed with an escape space 612 (or a relief space 612) into which a part of the arm engagement part 66 is moved when the arm engagement part 66 is pushed (pressed) by the release tool 80 that is inserted from the tool insertion hole 611b. Specifically, the escape space 612 is configured such that a part of the Y1-side end of the arm engagement part 66 on the Z2 side can move (escape) into the escape space 612. That is, the escape space 612 is configured such that a proximal end portion (Z2 side end portion) of the pair of restriction portions 662 of the arm engagement part 66, a proximal end portion (Z2 side portion) of the contact portion 665 of the arm engagement part 66, and the deformable portion 666 of the arm engagement part 66 can be moved into the escape space 612.

The escape space 612 is formed to be recessed toward the first surface 60a side (the Z2 side). That is, a part of the arm engagement part 66 can be escaped toward the first surface 60a side (Z2 side) opposite to the second surface 60b side (Z1 side) to which the surgical instrument 40 is attached.

As illustrated in FIGS. 4 and 5, the drive transmission members 62 are rotatably provided in the base body 61. Specifically, the drive transmission members 62 are provided rotatably about rotation axes extending in the Z direction. That is, the drive transmission members 62 are provided to be rotatable about the rotational axes thereof orthogonal to the first surface 60a and the second surface 60b. The drive transmission members 62 transmit rotations of the plural drive members 212 provided to the attachment portion 211 of the robot arm 21a to the plural driven members 44 provided to the surgical instrument 40. The number of the drive transmission members 62 corresponds to the number of the drive members 212 of the robot arm 21a and corresponds to the number of the driven members 44 of the surgical instrument 40. The plural drive transmission members 62 are respectively arranged in positions corresponding to the drive members 212 of the robot arm 21a and the driven members 44 of the surgical instrument 40.

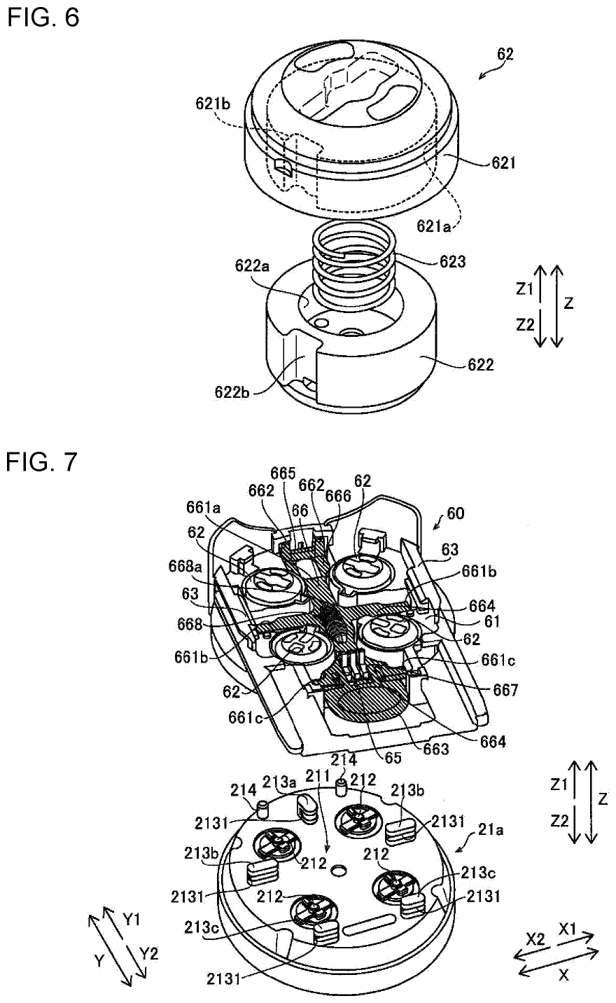

As illustrated in FIG. 6, each of the drive transmission member 62 includes a first member 621 and a second member 622. The second member 622 is provided movably with respect to the first member 621 with a bias member 623 interposed in between. The first member 621 includes a recess portion 621a and an engagement portion 621b. The recess portion 621a receives the second member 622 fitted thereto. The engagement portion 621b is engaged with the second member 622. The second member 622 includes a recess portion 622a in which the bias member 623 is accommodated and an engagement portion 622b to be engaged with the first member 621.

The first member 621 and the second member 622 are fitted to each other in the Z direction with the bias member 623 interposed in between. The first member 621 is positioned in the second surface 60b side (the Z1 side) with respect to the second member 622. The second member 622 is positioned in the first surface 60a side (the Z2 side). The bias member 623 biases the first member 621 toward the Z1 side with respect to the second member 622. For example, a spring constitutes the bias member 623.

As illustrated in FIG. 5, the guide rails 63 are provided on the second surface 60b of the adaptor 60. The guide rails 63 are provided to extend along the Y direction. The two guide rails 63 are provided to be opposed to each other in the X direction. The two guide rails 63 are provided substantially parallel to each other. The two guide rails 63 are provided corresponding to the two guide grooves 45 that are provided substantially parallel to each other on the attachment surface 40a of the surgical instrument 40. The guide rails 63 of the second surface 60b of the adaptor 60 are configured to receive the guide grooves 45 of the attachment surface 40a of the surgical instrument 40 to slide the surgical instrument 40 so as to guide the surgical instrument 40 to a position where the plural drive transmission members 62 provided to the adaptor 60 respectively correspond to the plural driven members 44 provided to the attachment surface 40a of the surgical instrument 40.

The precedence guide rail 64 is provided on the second surface 60b of the adaptor 60. The precedence guide rail 64 is provided to extend along the Y direction. The precedence guide rail 64 is provided between the two guide rails 63. The precedence guide rail 64 is formed to extend substantially parallel to the two guide rails 63. The precedence guide rail 64 is provided in the substantial center in the X direction of the second surface 60b. The precedence guide rail 64 is provided corresponding to the precedence guide groove 47 provided on the attachment surface 40a. Specifically, the precedence guide rail 64 guides the surgical instrument 40 before the two guide rails 63 guide the surgical instrument 40.

The electrode array 65 is connected to the electrode array 48 of the surgical instrument 40 and the robot arm 21a.

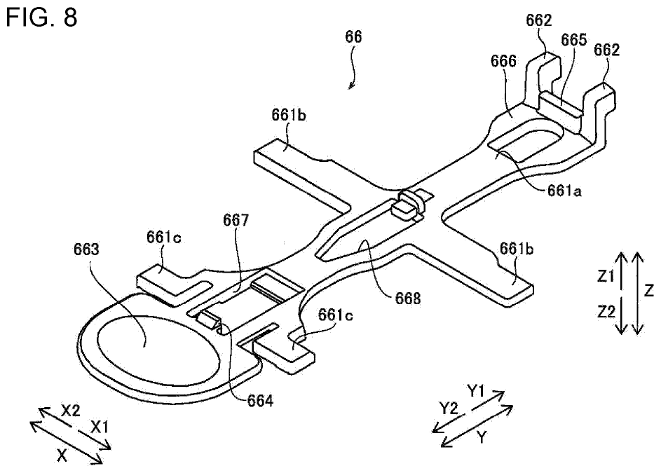

As illustrated in FIGS. 7 and 8, the arm engagement part 66 is engaged with the engagement projections 213a to 213c of the robot arm 21a. Specifically, the arm engagement part 66 is engaged with the engagement projections 213a to 213c that are inserted in the Z direction in the openings 67 provided in the first surface 60a. Further, the arm engagement part 66 includes the engagement portions 661a, 661b, and 661c, the restriction portions 662, the operating portion 663, a stopper 664, a contact portion 665, a deformable portion 666, an electrode arrangement portion 667, and a bias member arrangement portion 668. The arm engagement part 66 is biased toward the Y1 side by a bias member 668a arranged in the bias member arrangement portion 668. The number of the engagement portions 661b provided is two. The number of the engagement portions 661c provided is two. The number of the restriction portions 662 provided is two.

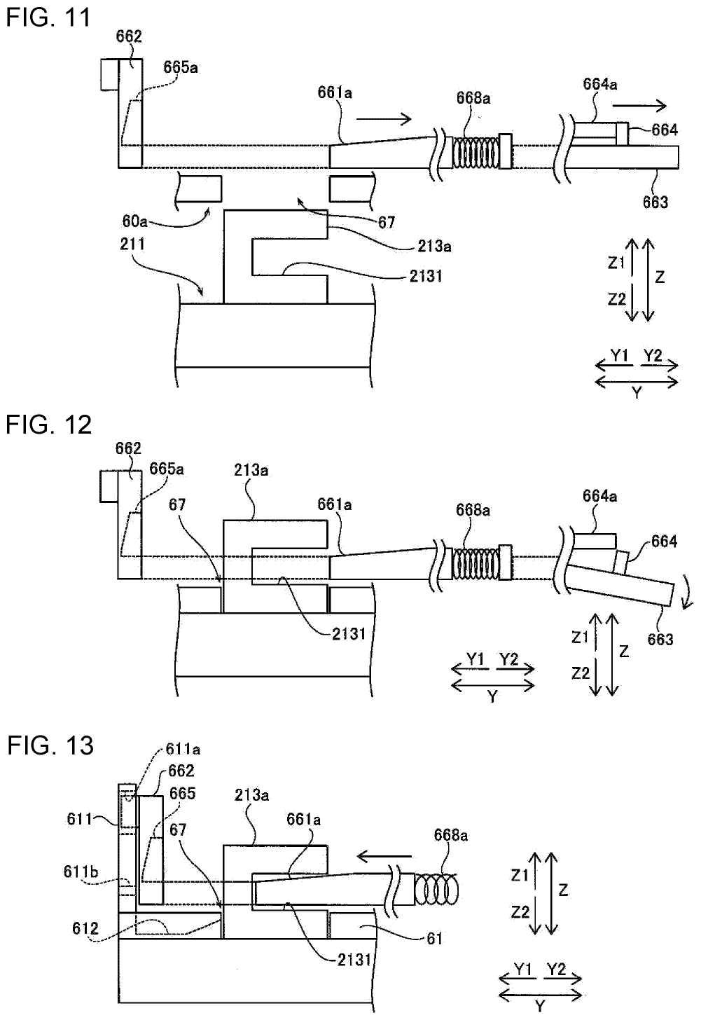

The engagement portions 661a to 661c of the arm engagement part 66 are configured to be movable with respect to the base body 61 in a predetermined direction. Specifically, the engagement portions 661a to 661c are movable with respect to the base body 61 in the Y direction. By moving the engagement portions 661a to 661c in the Y1 direction, the engagement portions 661a to 661c are engaged with the engagement projections 213a to 213c, respectively. On the other hand, by moving the arm engagement part 66 in the Y2 direction, the arm engagement part 66 are disengaged from the engagement projections 213a to 213c, respectively. The engagement portions 661a to 661c are movable between an engagement position (see FIG. 13) in which the engagement portions 661a to 661c are inserted into the grooves 2131 of the engagement projections 213a to 213c in the direction (Y direction) crossing the direction (Z direction) in which the first surface 60a and the attachment portion 211 of the robot arm 21a face each other and a disengagement position (see FIGS. 11 and 12) in which the engagement portions 661a to 661c are retracted from the grooves 2131 of the engagement projections 213a to 213c. The engagement portions 661a to 661c are engaged with the robot arm 21a at the engagement position.

As illustrated in FIG. 8, the restriction portions 662 are connected to the engagement portions 661a to 661c, the operating portion 663, and the contact portion 665. In the state where the surgical instrument 40 is attached to the second surface 60b of the adaptor 60, the restriction portions 662 are in contact with the surgical instrument 40 to restrict movements of the engagement portions 661a to 661c. With this configuration, in the state where the surgical instrument 40 is attached to the adaptor 60, it is possible to prevent the engagement portions 661a to 661c of the arm engagement part 66 from moving to the disengagement position. Therefore, it is possible to effectively prevent the adaptor 60 to which the surgical instrument 40 is attached from being unintentionally detached from the robot arm 21a.

Specifically, the restriction portions 662 are provided to be protruded toward the surgical instrument 40 side. The restriction portions 662 is configured such that in the state where the surgical instrument 40 is attached to the second surface 60b, the restriction portions 662 are in contact with the surgical instrument 40 and thus movements of the restriction portions 662 are restricted. That is, when the surgical instrument 40 is slid in the Y direction and thus attached to the adaptor 60, the Y1-side end of the housing 43 of the surgical instrument 40 comes into contact with the restriction portions 662. When the surgical instrument 40 is fixed to the adaptor 60, the surgical instrument 40 cannot move in the Y direction and thus the restriction portions 662 also cannot move in the Y direction.

The restriction portions 662 are composed of a pair of restriction portions 662 with the contact portion 665 provided between the pair of restriction portions 662. With this configuration, the movement of the engagement portions 661a to 661c can be regulated in a well-balanced manner by the pair of restriction portions 662 as compared with a case where a restriction portion(s) 662 is provided on only one side of the contact portion 665.

As illustrated in FIG. 7, the engagement projection 213a that is provided near the Y1-side end portion of the attachment portion 211 of the robot arm 21a is engaged with the engagement portion 661a that is provided near the Y1-side end portion of the arm engagement part 66. The two engagement projections 213b that are provided in the central portion of the attachment portion 211 of the robot arm 21a in the Y direction are respectively engaged with the two engagement portions 661b that are provided in the central portion of the arm engagement part 66 in the Y direction. The two engagement projections 213c that are provided near the Y2-side end portion of the attachment portion 211 of the robot arm 21a are engaged with the two engagement portions 661c that are provided near the Y2-side end portion of the arm engagement part 66.

The engagement portions 661a to 661c, the restriction portions 662, the operating portion 663, the stopper 664, the contact portion 665, and the deformable portion 666 are integrally formed. The arm engagement part 66 is made of, for example, resin material. The engagement portions 661a to 661c may be formed to be bendable in the direction from the engagement position to the disengagement position. In such a case, the bendability can absorb dimensional errors of the engagement portions 661a to 661c.

The operating portion 663 (a manipulation portion or a grab portion) is connected to the engagement portions 661a to 661c. The operating portion 663 is provided for moving the engagement portions 661a to 661c to the disengagement position against the biasing force of the bias member 668a. The operating portion 663 is operated by being pulled in the Y2 direction by an operator. The operating portion 663 is provided at an end portion of the arm engagement part 66 on the Y2 side. The operating portion 663 is provided for an operator to pull the operating portion 663 in the Y2 direction, to move the engagement portions 661a to 661c in the Y2 direction to release the engaged state of the engagement portions 661a to 661c. By moving the operating portion 663, the plural engagement portions 661a to 661c are integrally moved.

The stopper 664 stops the operating portion 663 so that the engagement portions 661a to 661c do not move from the disengagement position to the engagement position. With this configuration, after the operating portion 663 is moved in advance to the position where the engagement portions 661a to 661c of the adaptor 60 are located at the disengagement position, the position of the operating portion 663 is maintained by the stopper 664. Accordingly, the adaptor 60 can be easily attached to the robot arm 21a.

The contact portion 665 is provided for coming into contact with the release tool 80. Specifically, as illustrated in FIGS. 16 to 18, the contact portion 665 is configured such that the release tool 80 inserted from the tool insertion hole 611b comes into contact with the contact portion 665.

Here, in an embodiment described above, the arm engagement part 66 is configured such that (i) the contact portion 665 of the arm engagement part comes in contact with the release tool 80 when the release tool 80 is inserted through the tool insertion hole 611b into the base body, (ii) a part of the arm engagement part 66 is moved into the escape space 612 when the contact portion 665 is moved in the direction (Z2 direction) orthogonal to the insertion direction (Y2 direction) of the release tool 80, and (iii) the engagement portions 661a to 661c is moved, when the release tool 80 is further inserted, from the engagement position to the disengagement position to disengage the engagement portions 661a to 661c from the robot arm 21a.

As a result, by moving the release tool 80 in a certain direction (the insertion direction of the release tool 80), a part of the arm engagement part 66 is moved into the escape space and the engagement portions 661a to 661c are moved from the engagement position to the disengagement position. Accordingly, it is possible to simplify the work of detaching the surgical instrument 40 from the robot arm 21a. Even when the operating portion 663 of the arm engagement part 66 cannot be directly operated due to interference of the robot arm 21a or accessories of the surgical instrument 40, the arm engagement part 66 can be manipulated by the release tool 80 via the tool insertion hole 611b to disengage the robot arm 21a from the adaptor 60, and therefore, the surgical instrument 40 can be removed along with the adaptor 60 from the robot arm 21a.

That is, the arm engagement part 66 is configured such that the restriction on the movement of the engagement portions 661a to 661c toward the disengagement position by means of the restriction portions 662 is released, by moving the contact portion 665 in the direction (Z2 direction) orthogonal to the insertion direction (Y2 direction) of the release tool 80 while moving a part of the arm engagement part 66 to the escape space 612. With this configuration, when an operator wants to detach the adaptor 60 to which the surgical instrument 40 is attached from the robot arm 21a, the restriction on the movement of the engagement portions 661a to 661c by the restriction portions 662 can be released by inserting the release tool 80, so that the surgical instrument 40 together with the adaptor 60 can be removed from the robot arm 21a.

As illustrated in FIGS. 16 to 18, the contact portion 665 includes an inclined surface 665a at an area of the contact portion 665 where the release tool 80 contacts the contact portion 665, wherein the inclined surface is inclined with respect to the direction (Z direction) orthogonal to the insertion direction (Y2 direction) of the release tool 80. Specifically, the inclined surface 665a is inclined so as to approach the Y2 side as closer to the Z1 side. That is, the inclined surface 665a is inclined so as to go in the insertion direction (Y2 direction) of the release tool 80 as it goes in the direction (Z1 direction) opposite to the escape space 612. Thereby, the inclined surface 665a converts the pressing force in the insertion direction (Y2 direction) of the release tool 80 into the pressing force in the direction (Z2 direction) intersecting with the insertion direction (Y2 direction) of the release tool 80. Therefore, it is possible to easily move the contact portion 665 in the direction (Z2 direction) orthogonal to the insertion direction (Y2 direction) of the release tool 80 and easily move the part of the arm engagement part 66 to the escape space 612.

As illustrated in FIG. 8, the deformable portion 666 is connected to the restriction portions 662 and the contact portion 665. The deformable portion 666 is configured to be elastically (resiliently) deformable. Specifically, as illustrated in FIG. 17, the deformable portion 666 is configured to be elastically bent when the contact portion 665 is pressed by the release tool 80.

That is, the arm engagement part 66 is configured such that, when the deformable portion 666 of the arm engagement part 66 is elastically bent, the contact portion 665 of the arm engagement part 66 moves in the direction (Z2 direction) orthogonal to the insertion direction (Y2 direction) of the release tool 80, and the part of the arm engagement part 66 moves to the escape space 612. As a result, when the release tool 80 is inserted but the contact portion 665 is not pressed by the release tool 80, the deformable portion 666 is not elastically bent. Accordingly, it is possible to suppress the part of the arm engagement part 66 from unintentionally moving to the escape space 612.

As illustrated in FIG. 7, the electrode array 65 is arranged in the electrode arrangement portion 667. As illustrated in FIG. 8, the electrode arrangement portion 667 is composed of a through hole penetrating in the Z direction. The electrode arrangement portion 667 is provided so that the arm engagement part 66 does not interfere with the electrode array 65. That is, the electrode arrangement portion 667 is provided so that the electrode array 65 does not interfere with the arm engagement part 66 when the arm engagement part 66 is moved in the Y direction.

As illustrated in FIGS. 7 and 8, the bias member arrangement portion 668 is arranged substantially at the center of the adaptor 60 when viewed in the Z direction. The bias member 668a is arranged in the bias member arrangement portion 668. The bias member 668a biases the engagement portions 661a to 661c in the Y1 direction. That is, the bias member 668a biases the engagement portions 661a to 661c in a direction (Y1 direction) from the disengagement position toward the engagement position. For example, a spring constitutes the bias member 668a.

As illustrated in FIG. 4, the plural openings 67 are provided to the first surface 60a of the base body 61. Specifically, the adaptor 60 is fixed to the robot arm 21a by engagements at plural positions. For example, the number of the openings 67 provided is five. The plural openings 67 are arranged at substantially equal intervals along the outer peripheral side of the first surface 60a.

The positioning holes 68 are provided in the first surface 60a. The bosses 214 of the robot arm 21a are fitted to the positioning holes 68. The number of the positioning holes 68 provided is two or more (plural). The positioning holes 68 are provided near an end portion of the first surface 60a in the Y1 side.

The precedence guide portions 69 are configured, upon attaching the surgical instrument 40 to the adaptor 60, to guide the surgical instrument 40 before the guide rails 63 guides the surgical instrument 40. The precedence guide portions 69 are composed of a pair of precedence guide portions 69 provided substantially parallel to the first surface 60a and the second surface 60b and substantially parallel to each other at a predetermined interval in a direction (X direction) orthogonal to the direction for guiding the surgical instrument 40. A connecting portion 691 connects the pair of precedence guide portions 69, at the upstream side (Y2 side) ends of the precedence guide portions 69 in the slide insertion direction.

### (Attachment of Adaptor to Robot Arm)

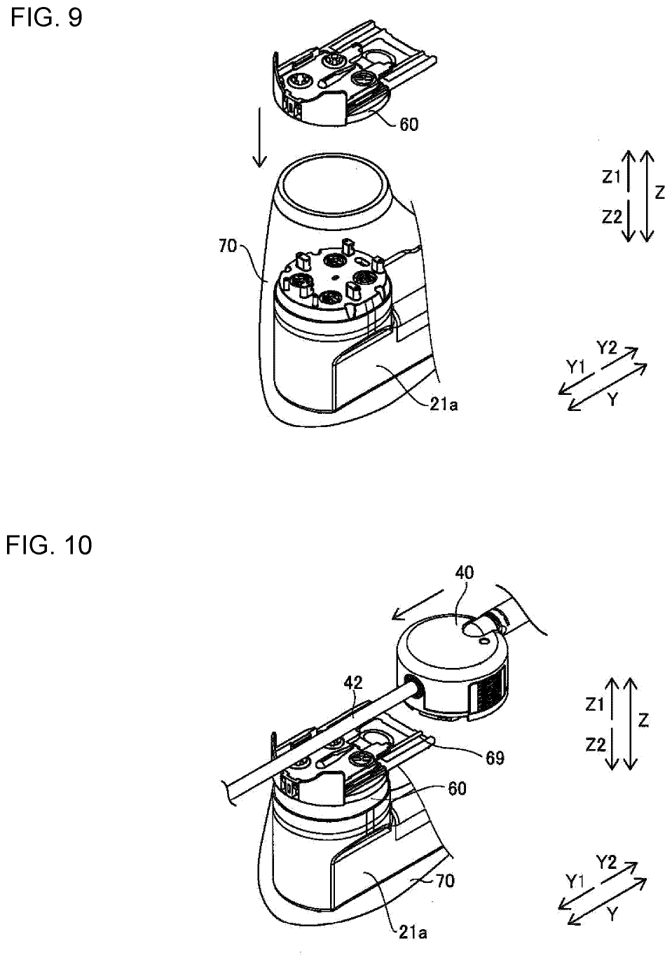

With reference to FIGS. 9 and 11 to 13, the attachment of the adaptor 60 to the robot arm 21a according to an embodiment is described.

As illustrated in FIG. 11, by operating the operating portion 663 connected to the engagement portions 661a to 661c, the operator moves the arm engagement part 66 to move the engagement portions 661a to 661c to the disengagement position against the bias force that biases the engagement portions 661a to 661c in the direction (Y1 direction) from the disengagement position toward the engagement position. As a result, the arm engagement part 66 provided on the second surface 60b side (the Z1 side) is moved so as to open the openings 67.

Further, at this position, the operating portion 663 is stopped by the stopper 664 so that the engagement portions 661a to 661c cannot move from the disengagement position to the engagement position. Specifically, the stopper 664 is engaged with a locking portion 664a

Next, as illustrated in FIG. 11, in the state where the operating portion 663 is stopped by the stopper 664 so that the engagement portions 661a to 661c cannot move from the disengagement position to the engagement position, the operator attaches the adaptor 60 to the robot arm 21 to make the adaptor 60 in contact with the robot arm 21a.

After that, as illustrated in FIG. 12, the operator releases the stopper 664 to release the stop of the movement of the operating portion 663 by means of the stopper 664. With this, by the biasing force of the bias member 668a, the arm engagement part 66 is moved to the engagement position so that the adaptor 60 (the engagement portions 661a, 661b, 661c) is engaged with the robot arm 21a (the engagement projections 213a, 213b, 213c), as illustrated in FIG. 13.

### (Attachment of Surgical Instrument to Robot Arm)

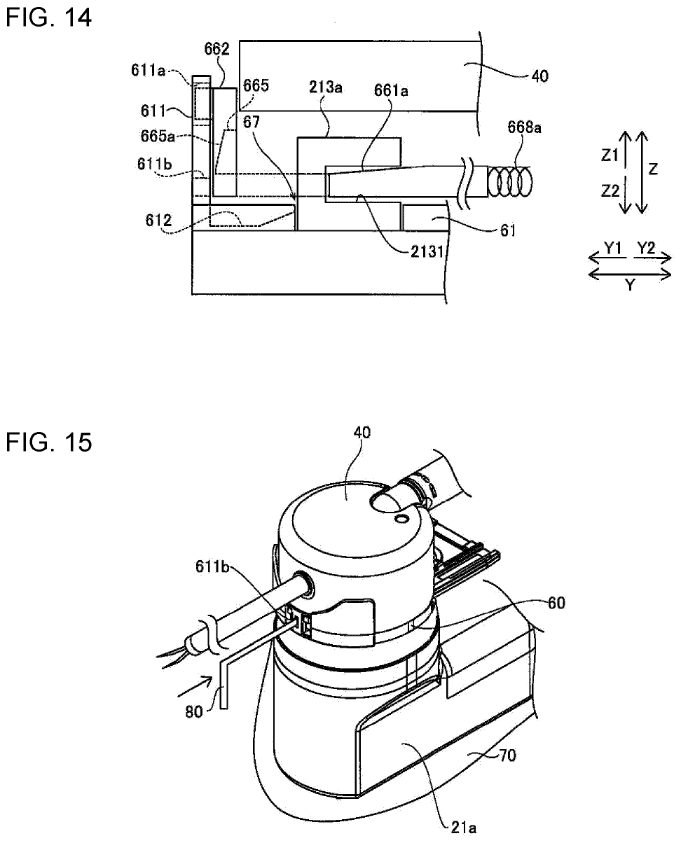

With reference to FIGS. 10 and 14, the attachment of the surgical instrument 40 to the robot arm 21a according to an embodiment is described.

First , the operator attaches the adaptor 60 to the robot arm 21a with the robot arm 21a being covered by the drape 70. Specifically, the operator moves the adaptor 60 in the Z direction with respect to the robot arm 21a so as to attach the adaptor 60 to the robot arm 21a. That is, the operator moves the adaptor 60 in the direction (Z direction) orthogonal to the first and second surfaces 60a and 60b so as to attach the adaptor 60 to the attachment portion 211 of the robot arm 21a.

Next, as illustrated in FIG. 10, the operator attaches the surgical instrument 40 to the adaptor 60 that is attached to the robot arm 21a. Specifically, the operator moves the surgical instrument 40 in the Y direction along the precedence guide portions 69, and the two guide rails 63 of the adaptor 60 so as to attach the surgical instrument 40 to the adaptor 60. In this way, the surgical instrument 40 is attached to the robot arm 21a via the adaptor 60.

As illustrated in FIG. 14, in the state where the surgical instrument 40 is attached to the adaptor 60 that is attached to the robot arm 21a, the surgical instrument 40 is in contact with the restriction portions 662 of the arm engagement part 66 of the adaptor 60, and thus the movement of the engagement portions 661a to 661c of the arm engagement part 66 is restricted.

When the operator wants to detach the surgical instrument 40 from the robot arm 21a, the operator slides the surgical instrument 40 in the Y2 direction while pressing the buttons 461 of the movable members 46 of the surgical instrument 40, so as to detach the surgical instrument 40 from the adaptor 60.

When the operator wants to detach the adaptor 60 from the robot arm 21a, in the state where the surgical instrument 40 is detached from the adaptor, the operator pulls the operating portion 663 of the arm engagement part 66 in the Y2 direction, so as to release the engagement between the adaptor 60 and the robot arm 21a. In this state, the operator moves the adaptor in the Z1 direction, so that the adaptor 60 is detached from the robot arm 21a.

(Detachment of Surgical Instrument from Robot Arm using Release Tool)

With reference to FIGS. 14 to 18, the detachment of the surgical instrument 40 from the robot arm 21a using the release tool 80 according to an embodiment is described.

As illustrated in FIGS. 15 and 16, the operator inserts the release tool 80 through the tool insertion hole 611b, and brings the release tool 80 inserted from the tool insertion hole 611b into contact with the contact portion 665 of the arm engagement part 66.

Next, as illustrated in FIG. 17, the operator presses the release tool 80 against the contact portion 665 in the insertion direction (Y2 direction) of the release tool 80. As a result, the inclined surface 665a of the contact portion 665 is pressed in the Y2 direction, and thus the contact portion 665 is moved in the Z2 direction. That is, the contact portion 665 moves in the direction (Z2 direction) orthogonal to the insertion direction (Y2 direction) of the release tool 80, and a part of the arm engagement part 66 moves to the escape space 612 of the base body 61. As a result, the restriction portion 662 connected to the contact portion 665 also moves in the Z2 direction, and the restriction portion 662 and the surgical instrument 40 are offset from each other. That is, the restriction on the movement of the restriction portion 662 by the surgical instrument 40 is released.

Next, the operator further inserts the release tool 80, as illustrated in FIG. 18. As a result, the engagement portions 661a to 661c of the arm engagement part 66 are moved from the engagement position to the disengagement position. Thus, the engagement between the engagement portions 661a to 661c and the robot arm 21a is released. After that, the operator moves the surgical instrument 40 together with the adaptor 60 in the Z1 direction, so as to detach the surgical instrument 40 together with the adaptor from the robot arm 21a. In other words, in an embodiment described above, the arm engagement part 66 is configured, upon inserting the release tool 80 through the tool insertion hole 611b and pressing the release tool 80 that is inserted through tool insertion hole 611b against the contact portion 665 of the arm engagement part in the insertion direction (Y2 direction) of the release tool 80, to move the contact portion 665 in the direction (Z2 direction) orthogonal to the insertion direction (Y2 direction) of the release tool 80 while moving a part of the arm engagement part 66 into the escape space 612, and is configured to, upon further pressing the release tool in the insertion direction (Y2 direction), to move the engagement portions 661a to 661c from the engagement position to the disengagement position, which disengages the engagement portions 661a to 661c from the robot arm 21a.

(Modifications)

Note that one or more embodiments disclosed herein should be considered as exemplary in all respects and do not limit the invention. The scope of the invention is indicated by claims, not by explanation of one or more embodiments described above, and includes equivalents to the claims and all alterations (modification) within the same.

For example, in one or more embodiments described above, the case has been described in which the adaptor and the attachment portion of the robot arm are engaged with each other by means of the engagement portions at the five locations. However, the invention is not limited thereto. In the invention, the adaptor and the attachment portion of the robot arm are engaged with each other by means of engagement portions at plural locations more than five or less than five.

In one or more embodiments described above, the case has been described in which the contact portion includes the inclined portion. However, the invention is not limited thereto. In the invention, the release tool may have, in an area where the release tool contacts the contact portion, an inclined surface that is inclined with respect to a direction orthogonal to the insertion direction of the release tool. Further, at least one of the contact portion and the release tool may have an inclined portion.

Further, in one or more embodiments described above, the case has been described in which the adaptor is formed in a substantially circular shape in the plan view. However, the invention is not limited thereto. In the invention, the adaptor may not be formed in such a substantially circular shape in the plan view. For example, the adaptor may be formed in a rectangular shape in the plan view.

Further, in one or more embodiments described above, the case has been described in which, in the state where the surgical instrument is attached to the adaptor, the movement of the arm engagement part is restricted by the contact of the surgical instrument. However, the invention is not limited thereto. In the invention, in the state where the surgical instrument is attached to the adaptor, the movement of the arm engagement part may be restricted by a portion or a part other than the surgical instrument.

In one or more embodiments described above, the case has been descried in which the number of the drive transmission members provided to the adaptor is four. However, the invention is not limited thereto. In the invention, the adaptor may include a plurality of drive transmission members more than four or less than four.

In one or more embodiments described above, the case has been described in which the surgical instrument is attached or detached by being slid and moved along the second surface of the adaptor. However, the invention is not limited thereto. In the invention, the surgical instrument may be attached or detached by moving the surgical instrument in a direction other than the direction along the second surface of the adaptor. For example, the surgical instrument may be attached or detached by moving the surgical instrument with respect to the adaptor in a direction orthogonal to the second surface.

In one or more embodiments described above, the case has been described in which the adaptor and the drape are provided independently of each other. However, the invention is not limited thereto. In the invention, the adaptor and the drape may be integrally provided.

1. An adaptor to be detachably attached to a robot arm for transmitting a driving force from the robot arm to a surgical instrument, the adaptor comprising:
a base body including a first surface to be attached to the robot arm and a second surface to which the surgical instrument is to be attached, the first surface of the base body being formed with an opening; and
an arm engagement part including an engagement portion configured to be movable between an engagement position corresponding to the opening of the first surface and a disengagement position retracted from the engagement position and to be engaged with the robot arm at the engagement position, and a contact portion with which a release tool comes in contact, wherein
the base body is formed with a tool insertion hole through which the release tool is to be inserted and an escape space into which a part of the arm engagement part is to be moved by the release tool that is inserted through the tool insertion hole and is in contact with the arm engagement part, and
the arm engagement part is configured such that the contact portion of the arm engagement part comes in contact with the release tool when the release tool is inserted through the tool insertion hole, the part of the arm engagement part is moved into the escape space when the contact portion is moved in a direction orthogonal to an insertion direction of the release tool, and the engagement portion of the arm engagement part is moved from the engagement position to the disengagement position when the release tool is further inserted, so as to disengage the engagement portion of the arm engagement part from the robot arm.

2. The adaptor according to claim 1, wherein
at least one of the contact portion and the release tool includes, in an area where the release tool contacts the contact portion, an inclined surface that is inclined with respect to the direction orthogonal to the insertion direction of the release tool.

3. The adaptor according to claim 1, wherein
the arm engagement part includes a restriction portion configured, in a state where the surgical instrument is attached to the second surface of the adaptor, to be in contact with the surgical instrument and thus to restrict movement of the engagement portion from the engagement position to the disengagement position, and
the arm engagement part is configured, when the contact portion is moved in the direction orthogonal to the insertion direction of the release tool, to move the part of the arm engagement part into the escape space, so as to release restriction of the movement of the engagement portion to the disengagement position by the restriction portion.

4. The adaptor according to claim 3, wherein
the restriction portion comprises a pair of restriction portions with the contact portion located between the pair of restriction portions.

5. The adaptor according to claim 1, wherein
the tool insertion hole is formed in a slit shape extending in a direction in which the first surface and the second surface are opposed to each other.

6. The adaptor according to claim 1, wherein
the escape space is formed to be recessed toward the first surface.

7. The adaptor according to claim 1, wherein
the arm engagement part further comprises: a bias member configured to bias the engagement portion in a direction from the disengagement position toward the engagement position; an operating portion connected to the engagement portion for moving the engagement portion to the disengagement position against a biasing force of the bias member; and a stopper configured to stop the operating portion to prevent the engagement portion from moving from the disengagement position to the engagement position.

8. The adaptor according to claim 1, wherein
the arm engagement part includes a deformable portion connected to the contact portion and being elastically deformable, and
the arm engagement part is configured such that, when the deformable portion of the arm engagement part is elastically deformed, the contact portion of the arm engagement part moves in the direction orthogonal to the insertion direction of the release tool and the part of the arm engagement part moves to the escape space.

9. The adaptor according to claim 1, further comprising:
plural drive transmission members rotatably provided to the base body, for transmitting rotations of plural drive members provided to the robot arm to plural driven members provided to the surgical instrument.

10. The adaptor according to claim 1, further comprising:
the adaptor is attached to the robot arm via a drape.

11. A method of detaching an adaptor from a robot arm, wherein the adaptor includes a base body including a first surface to be attached to the robot arm and a second surface to which a surgical instrument is to be attached, and an arm engagement part including an engagement portion to be engaged with the robot arm, and wherein the adaptor is configured to be detachably attached to the robot arm and transmit a driving force from the robot arm to the surgical instrument, the method comprising:
inserting a release tool for releasing engagement between the robot arm and the engagement portion of the arm engagement part of the adaptor into a tool insertion hole provided to the base body, so as to bring the release tool in contact with a contact portion of the arm engagement part;
moving the contact portion in a direction orthogonal to an insertion direction of the release tool so as to move a part of the arm engagement part to an escape space in the base body; and
further inserting the release tool, to move the engagement portion of the arm engagement part from an engagement position to a disengagement position so as to release the engagement between the robot arm and the engagement portion of the arm engagement part.

12. A robotic surgical system, comprising:
a surgical instrument; and
an adaptor detachably attachable to a robot arm and configured to transmit a driving force from the robot arm to the surgical instrument, wherein
the adaptor comprises: a base body including a first surface that is to be attached to the robot arm and formed with an opening and a second surface to which the surgical instrument is to be attached; and an arm engagement part including an engagement portion configured to be movable between an engagement position corresponding to the opening of the first surface and a disengagement position retracted from the engagement position and to be engaged with the robot arm at the engagement position, and a contact portion with which a release tool comes in contact,
the base body includes a tool insertion hole through which the release tool is to be inserted and an escape space into which a part of the arm engagement part is to be moved by the release tool that is inserted through the tool insertion hole and is in contact with the arm engagement part, and
the arm engagement part is configured such that the contact portion of the arm engagement part comes in contact with the release tool when the release tool is inserted through the tool insertion hole, the part of the arm engagement part is moved into the escape space when the contact portion is moved in a direction orthogonal to an insertion direction of the release tool, and the engagement portion of the arm engagement part is moved from the engagement position to the disengagement position when the release tool is further inserted, so as to disengage the engagement portion of the arm engagement part from the robot arm.

13. The robotic surgical system according to claim 12, wherein
at least one of the contact portion and the release tool includes, in an area where the release tool contacts the contact portion, an inclined surface that is inclined with respect to the direction orthogonal to the insertion direction of the release tool.

14. The robotic surgical system according to claim 12, wherein
the arm engagement part includes a restriction portion configured, in a state where the surgical instrument is attached to the second surface of the adaptor, to be in contact with the surgical instrument and thus to restrict movement of the engagement portion from the engagement position to the disengagement position, and
the arm engagement part is configured, when the contact portion is moved in the direction orthogonal to the insertion direction of the release tool, to move the part of the arm engagement part into the escape space, so as to release restriction of the movement of the engagement portion to the disengagement position by the restriction portion.

15. The robotic surgical system according to claim 14, wherein
the restriction portion comprises a pair of restriction portions with the contact portion located between the pair of restriction portions.

16. The robotic surgical system according to claim 12, wherein
the tool insertion hole is formed in a slit shape elongated in a direction in which the first surface and the second surface are opposed to each other.

17. The robotic surgical system according to claim 12, wherein the escape space is recessed toward the first surface.

18. The robotic surgical system according to claim 12, wherein
the arm engagement part further comprises: a bias member configured to bias the engagement portion in a direction from the disengagement position toward the engagement position; an operating portion connected to the engagement portion for moving the engagement portion to the disengagement position against a biasing force of the bias member; and a stopper configured to stop the operating portion to prevent the engagement portion from moving from the disengagement position to the engagement position.

19. The robotic surgical system according to claim 12, wherein
the arm engagement part includes a deformable portion connected to the contact portion and being elastically deformable, and
the arm engagement part is configured such that, when the deformable portion of the arm engagement part is elastically deformed, the contact portion of the arm engagement part moves in the direction orthogonal to the insertion direction of the release tool and the part of the arm engagement part moves to the escape space.

20. The robotic surgical system according to claim 12, further comprising
plural drive transmission members rotatably provided to the base body, for transmitting rotations of plural drive members provided to the robot arm to plural driven members provided to the surgical instrument.
